(19) United States
(12) Patent Application Publication
(10) Pub. No.: US 2010/0111985 A1
(43) Pub. Date: May 6, 2010

(54) VACCINE COMPOSITIONS AND METHODS OF USE

(75) Inventors: Gunter SCHWAMBERGER, Salzburg (AT); Thomas E. Wagner, Greenville, SC (US)

Correspondence Address: FOLEY AND LARDNER LLP;SUITE 500, 3000 K STREET NW, WASHINGTON, DC, 20007, US

(73) Assignee: ORBIS Health Solutions, LLC

(21) Appl. No.: 12/610,964

(22) Filed: Nov. 2, 2009

Related U.S. Application Data
(63) Continuation-in-part of application No. 12/149,097, filed on Apr. 25, 2008.
(60) Provisional application No. 60/907,977, filed on Apr. 25, 2007.
(60) Provisional application No. 60/924,868, filed on Jun. 4, 2007.

(51) Int. Cl. A61K 39/00; A61P 37/04

(52) U.S. Cl. 424/184.1

(57) ABSTRACT

Described are a method and a composition for delivery of a protein to an antigen presenting cell. The composition is composed of a polypeptide component, a buffering component and a particle to be phagocytized. In one embodiment, the antigen presenting cell is aa macrophage or a dendritic cell and the particle to be phagocytized is from a natural source, such as from a microbial source. The composition itself, or cells pretreated with the composition, are useful for strategies in vaccine development.

### CROSS-REFERENCE TO RELATED PATENT APPLICATIONS

This application is a continuation in part application of U.S. application Ser. No. 12/149,097, filed Apr. 25, 2008, which claims priority to U.S. Provisional Application No. 60/907,977, filed Apr. 25, 2007, and U.S. Provisional Application No. 60/924,868, filed Jun. 4, 2007, and are incorporated herein by reference in their entirety.

### FIELD OF THE INVENTION

The present invention generally relates to compositions and methods for delivering a protein, peptide, epitope, or antigen to an antigen presenting cell, such as a macrophage or dendritic cell. The compositions and methods disclosed herein are particularly useful in making prophylactic and therapeutic vaccines.

### BACKGROUND OF THE INVENTION

Antigen presenting cells, including macrophages and other cells of the mononuclear phagocyte system actively phagocytose particles and play a central role in the immune response. Macrophages are cells within the tissues that are derived from monocytes. These monocytes/macrophages phagocytose microbes, for example, which are then digested to smaller antigenic portions in the lysosome/phagosome. The resultant antigens are cycled back to the surface for presentation to the humoral and cellular arms of the immune system. Accordingly, monocytes/macrophages are of particular interest because they play an important role in both nonspecific and specific defenses in the host against pathogens.

Dendritic cells are also antigen presenting cells that express MHC class I and class II molecules. An ideal vaccine mimics the rapid uptake and transfer of pathogenic structures without actually establishing an infection and without causing suppression of the MHC class I pathway. For this purpose, the present invention relates to a composition comprising a modified particle to be phagocytosed that is avidly and specifically taken up by professional phagocytic cells, and methods for delivering cargo molecules to the cytoplasm of antigen presenting cells for presentation on MHC class I molecules.

Before the present invention, delivery of exogenous antigens, peptides, or proteins to an antigen presenting cell for presentation on class I MHC molecules was difficult because following degradation in vesicular intracellular compartments, such antigens would be loaded on MHC class II molecules for presentation. Indeed, the only antigens that normally activate the MHC class I pathway are those that are derived from cytosolic antigens (e.g., endogenously produced within an antigen presenting cell). Although dendritic cells can exhibit “cross presentation” phenomena, whereby dendritic cells present exogenous antigens on a class I molecule, the localized concentration of soluble, exogenous antigen must be very high (approximately 100 or even 1000 fold higher than the present invention) for such cross presentation to occur and is therefore inefficient. On the other hand, the compositions of the present invention can efficiently deliver exogenous proteins, epitopes, antigens, and peptides, for presentation on class I molecules with only a very low amount of exogenous material.

Thus, while some proteins that escape the phagosome and enter into the cytosol of an antigen presenting cell could also activate the MHC class I pathway, a cost effective and efficient delivery method of exogenous proteins, epitopes, antigens and/or peptides for association with MHC class I molecules could not be purposefully accomplished before the present invention.

### SUMMARY OF THE INVENTION

One embodiment of the present invention is an antigenic composition comprising (i) a polypeptide component, (ii) a buffering component, and (iii) a particle that can be phagocytosed, wherein the polypeptide component or a fragment thereof is ultimately presented on a class I MHC molecule.

Another embodiment of the invention is a method for efficient delivery of a polypeptide component to an antigen presenting cell comprising administering a composition comprising (i) a polypeptide component, (ii) a buffering component, and (iii) a particle that can be phagocytosed, wherein the polypeptide component, following administration, enters the cytosol from an endocytotic vesicle, and the polypeptide or a fragment thereof is presented on a MHC class I molecule.

Also described herein is a composition for exogenous antigen presentation on class I MHC molecules comprising (i) a polypeptide component, (ii) a buffering component, and (iii) a particle that can be phagocytosed, and a vaccine composition comprising (i) a polypeptide component, (ii) a buffering component, and (iii) a particle that can be phagocytosed, wherein the polypeptide component or a fragment thereof is ultimately presented on a class I MHC molecule. The polypeptide component is also delivered in an amount sufficient to provoke a CD8 T cell response, in the case of dendritic cells.

In the compositions and methods of the present invention, the particle that can be phagocytosed is a biodegradable particle, such as zymosan particle, chitin particle, agarose particle or sepharose particle, and the buffering component can be any buffer with a buffering capacity in the range of about pH 6 to about pH 8, such as RCONHNH₂, polyethyleneimine, oligohistidine, oligoornithine, and oligolysine.

### BRIEF DESCRIPTION OF THE DRAWINGS

FIG. 1. Antigenic stimulation of MHC-I (H-2K^{b}) restricted B3Z hybridoma reporter cells specific for the internal OVA epitope SINFEKL in the absence or presence of IC-21 (H-2K^{b}) antigen presenting cells.

### DETAILED DESCRIPTION OF THE INVENTION

### Introduction

The present invention provides a composition for delivery to an antigen presenting cell, and a related method of use. The composition according to the present invention comprises a polypeptide component, attached to a particle that can be phagocytosed and a buffering component. The present invention takes advantage of the phagocytic property of certain antigen presenting cells in that the polypeptide component is provided on a substrate that “looks” like a microbe. Thus, the particle attracts phagocytic cells.

The compositions of the present invention do not contain liposomes (vesicles with an aqueous interior enclosed by one or more phospholipid bilayers), nor are the compositions disclosed herein taken up by macrophages, dendritic, or other antigen presenting cells based on an agent that permeabilizes the extracellular membrane of these cells. The compositions of the present invention also do not utilize haemolysin to permeabilize the phagosomal membrane.

### Particle to be Phagocytosed

In one embodiment of the present invention, the particle to be phagocytosed (“particle”) is a biodegradable particle, such as one derived from natural sources. In one embodiment, the particle to be phagocytosed is of microbial origin, and is preferably a particle from a yeast cell wall. In another embodiment, the yeast cell wall particle is a zymosan particle. Zymosan (also referred to as Zymosan A) is commercially available from various companies such as Sigma-Aldrich. Natural particles, such as zymosan, are better tolerated by macrophages, for example, than magnetic beads and particles from other sources.

Zymosan is an insoluble polysaccharide component of yeast cell wall. Prior publications uncovered zymosan's involvements in (i) induction of the release of cytokines or proinflammatory cytokines, (ii) induction of protein phosphorylation and inositol phosphate formation, (iii) arachidonate mobilization, (iv) activation of the alternative complement pathway; and (v) raise of cyclin D2 levels, suggesting a role of cyclin D2 in macrophage activation (Miyasato et al., Int. Arch. Allergy Immunol. 104: 24-26, 1994). For example, it has been reported that zymosan particles are capable of inducing inflammatory signals in macrophages through Toll-like receptors, e.g. TLR2 and TLR6, and dectin-1, which is a receptor that binds β-glucans and is important for macrophage phagocytosis. Zymosan is also involved in inducing inflammatory responses, such as TNF-α production and NF-κB activation in macrophages (Underhill, Journal of Endotoxin Research, 9: 176-180, 2003; Sato et al., J. Immunol., 171: 417-425, 2003; Dillon et al., J. Clin. Invest. 116: 916-928, 2006).

A preferred size for the particle that can be phagocytosed is one that approximates the size of microbial structures that cells of the mononuclear phagocyte system and other phagocytic cells typically ingest. In one embodiment, the particle will be about 0.05 to about 5.0 μm, about 0.05 to about 2.5 μm, about 0.1 to about 2.5 μm, about 1.0 to about 2.5 μm, about 1.0 to about 2.0 μm, or about 1.0 to about 1.5 μm. The term “about” in this context refers to ±0.25 μm. Zymosan is typically about 2.0 μm in size.

Although the particle that can be phagocytosed is not limited by any particular size, a preferred size for the particle that can be phagocytosed is one that approximates the size of microbial structures that cells of the mononuclear phagocyte lineage (e.g., monocytes, macrophages, dendritic cells, dendritic cell precursors (immature dendritic cells), or other antigen presenting cells, typically ingest. In one embodiment, the particle will be about 0.05 to about 5.0 μm, about 0.05 to about 2.5 μm, about 0.1 to about 2.5 μm, about 1.0 to about 2.5 μm, about 1.0 to about 2.0 μm, or about 1.0 to about 1.5 μm. The term “about” in this context refers to ±0.25 μm. Zymosan is typically about 2.0 μm in size.

The particle that can be phagocytosed is also not limited by shape or material. In general, the particle can be of any shape or material that allows the composition of the present invention to be phagocytized by cells of the mononuclear phagocyte system, such as monocytes/macrophages, dendritic cells, immature dendritic cells (have a high capacity for phagocytosis, then undergo maturation), or other phagocytic cells. For example, in addition to zymosan, the particle to be phagocytosed can be made of chitin, a synthetic beta glucan polymer, agarose, sepharose, etc., so long as the particle contains a carbohydrate or other moiety that permits attachment of the polypeptide component of the present invention.

### Polypeptide Component

The polypeptide component of the present invention is attached to the particle to be phagocytosed. Following administration of the polypeptide component attached to the particle, the polypeptide component, or fragment thereof is released from the particle, enters the cytosol from an endocytotic vesicle (e.g., phagosome) and, as a result of normal “processing”, is presented on a MHC class I molecule.

The polypeptide component for delivery to a phagocytic cell (“cargo”) comprises an amino acid sequence, and can be at least one peptide such as at least one epitope (approximately 8-12 amino acids in length), at least one small peptide (e.g., approximately <30 amino acids in length), at least one large peptide (approximately >30 amino acids in length), at least one full length protein, or a combination thereof. For example, the cargo can be composed of at least one tumor antigen, protein, protein fragment or a combination thereof The cargo may also be a tumor cell lysate; the large antigen capacity of the particle to be phagocytosed allows for the coupling of complex protein mixtures derived from a patient's lysed tumor tissues. The polypeptide component is an exogenous polypeptide (i.e., exogenous relative to the phagocytic cell).

In one embodiment, suitable cargo for use in the present invention can be allergens, viral antigens, bacterial antigens and antigens derived from parasites. Preferred antigens include tumor associated antigens, with which the artisan will be familiar (e.g., carcinoembryonic antigen, prostate-specific membrane antigen, melanoma antigen, adenocarcinoma antigen, leukemia antigen, lymphoma antigen, sarcoma antigen, MAGE-1, MAGE-2, MART-1, Melan-A, p53, gp100, antigen associated with colonic carcinoma, antigen associated with breast carcinoma, Muc1, Trp-2, telomerase, PSA and antigen associated with renal carcinoma). Whole inactivated viruses, portions of the virus, and viral antigens are also suitable polypeptide components for the present invention. In another embodiment, viral antigens include HIV, EBV, Herpes virus, and a linear gp41 epitope insertion (LLELDKWASL), which has been identified as a useful construct for improving HIV-1 Env immunogenicity (Liang, et al., Vaccine, 16; 17(22):2862-72, July 1999).

### Buffering Component

The compositions of the present invention employ a buffering component, which allows the polypeptide component to evade the lysosome and enter the cytosol of the antigen presenting cell.

More specifically, when a cell of the mononuclear phagocyte system, such as a monocyte (or monocyte derived cell), macrophage, dendritic cell or dendritic cell precursor, ingests an antigen, a phagocytic vesicle (phagosome) which engulfs the antigen is formed. Next, a specialized lysosome contained in the phagocytic cell fuses with the newly formed phagosome. Upon fusion, the phagocytosed antigen is exposed to several highly reactive molecules as well as a concentrated mixture of lysosomal hydrolases. These highly reactive molecules and lysosomal hydrolases digest the contents of the phagosome/lysosome. Therefore, by covalently attaching a buffering component to the particle to be phagocytosed, the polypeptide component that is also attached to the particle escapes digestion by the materials in the phagosome/lysosome and enters the cytoplasm of the phagocytic cell because the buffering component is localized in the phagosome and causes a continued influx of protons, accompanied by chloride ions into the phagosome, and therefore osmotic swelling and ultimately rupture of the endosomal membrane. When the phago some bursts, fusion between the phagosome and lysosome does not occur.

Accordingly, the inventors of the present invention surprisingly discovered how to efficiently deliver an exogenously produced antigen, epitope, protein, peptide or other amino acid sequence 8 amino acids in length or greater, even in very small amounts, to a phagocytic cell, such as a cell of the mononuclear phagocyte system (e.g., monocyte/macrophage, dendritic cell, dendritic cell precursor), for cell surface expression by class I MHC molecules.

In one embodiment, the buffering component has a buffering capacity in a pH range of about pH 6 to about pH 8. An example of a buffering component suitable for use in the present invention is RCONHNH₂. Another example of a buffering component is an oligo-histidine, oligo-lysine, polyamine, polyethylenimine (e.g., a low molecular weight PEI, either branched or linear), and oligo-ornithine. Several exemplary buffers can be found in a “pKa data compilation by R. Williams”, available at http://research.chem.psu.edu/brpgroup/pKa_compilation.pdf, which is incorporated by reference herein in its entirety. The buffering component does not form a complex with the polypeptide component as one of the buffering components exemplified herein can form with nucleic acid. The buffering component may, however, be used as a way to attach the polypeptide component. But in that way, the “buffering component” does not have a buffering capacity in a pH range of about 6-8 and is therefore not acting as a buffer; it is acting as a chemical linkage instead.

In addition to the buffering component described herein, the compositions of the present invention may optionally contain an additional component that evades the degrading environment of the phagosome/lysosome. Such an additional “lysosome evading component” can be added to the compositions of the present invention and includes any number of amino acids, carbohydrates, lipids, fatty acids, biomimetic polymers, microorganisms and combinations thereof.

Preferred lysosome evading components include proteins, viruses or parts of viruses. The adenovirus penton protein, for example, is a well known complex that enables the virus to evade/disrupt the lysosome/phagosome. Thus, either the intact adenovirus or the isolated penton protein, or a portion thereof (see, for example, Bal et al., Eur J Biochem 267:6074-81 (2000)), can be utilized as the lysosome evading component. Fusogenic peptides derived from N-terminal sequences of the influenza virus hemagglutinin subunit HA-2 may also be used as the lysosome evading component (Wagner, et al., Proc. Natl. Acad. Sci. USA, 89:7934-7938, 1992).

Other preferred lysosome evading components include biomimetic polymers such as Poly (2-propyl acrylic acid) (PPAAc), which has been shown to enhance cell transfection efficiency due to enhancement of the endosomal release of a conjugate containing a plasmid of interest (see Lackey et al., Abstracts of Scientific Presentations: The Third Annual Meeting of the American Society of Gene Therapy, Abstract No. 33, May 31, 2000-Jun. 4, 2000, Denver, Colo.). Examples of other lysosome evading components envisioned by the present invention are discussed by Stayton, et al. J. Control Release, 1;65(1-2):203-20, 2000.

### Method for Attaching the Polypeptide Component to the Particle to be Phagocytosed

Any number of methods can be used to attach the cargo to the particle to be phagocytosed.

Attachment of the components discussed above to the particle to be phagocytosed can be done by any number of means. In principle, the target protein can be linked to a particle to be phagocytosed, such as a biodegradable particle, either directly or indirectly. Direct attachment, for instance, typically requires the biodegradable particle and polypeptide component to present appropriate functional groups such that a direct link between the particle and polypeptide component can be formed via reaction of these groups, and then the polypeptide component can be readily cleaved off so as to release the polypeptide component. For example, the polypeptide component may contain a sulfhydryl group that ultimately allows for attachment to the particle, or can be modified by the addition of a cysteine residue at its N or C terminus.

Indirect methods for attachment typically utilize well-known and rich chemistry of linkers suitable for this purpose and, as with direct attachment, the methods establish a cleavable bond that gives rise to free polypeptide component. In either case, the particle to be phagocytosed possesses free amino groups or it can be modified with reagents to present such groups, as described in more detail below.

In particular, without wishing to be bound by any particular theory, the inventors believe that such linking moieties are useful not only for presenting appropriate combinations of functional groups for linking the particle and protein, but also for possessing buffering properties in a physiological environment. The latter property is believed to allow the linked protein and particle to withstand the otherwise destructive action of intracellular lysosomes and/or phagosomes, thereby preserving the protein in its entirety until it can be cleaved from the particle-linker. Suitable buffers in this regard should confer a pH of about 6- to about 8 to the final product.

An illustrative biodegradable particle in this context is zymosan because it presents convenient functional groups for modification, although any other biodegradable particle that similarly presents suitable functional groups can be adapted to the synthetic methodologies described herein. As described above, direct or indirect attachment methods are well-known. In some embodiments, the particle contains amino groups that can be incorporated directly into the synthetic schemes below. In other embodiments that are explicitly set forth below, the particles do not present amino groups but can be modified to exploit advantageous properties of one or more linking moieties that do contain amino groups.

To illustrate, zymosan was reacted with a source of periodate, such as sodium periodate, to yield aldehyde moieties in intermediate A as shown in reaction (i) below:

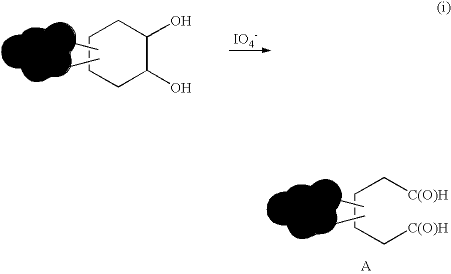

Many particles to be phagocytosed are suitable for use in the invention. The examples herein utilize zymosan, but any number of other well-known particles can be used such as, for instance, agarose.

The next steps introduce appropriate linkers and buffers consistent with the general requirements discussed above. In general, a linker can also possess a buffering property, or a linker and buffer can be distinct moieties. In addition, the basic chemical requirement is that the combination of linker and buffer possess compatible functional groups so as to ultimately present a disulfide moiety that is highly useful for attaching an appropriately modified protein, as described in more detail below.

Thus, in one alternative, the aldehyde groups in intermediate A were reacted with a convenient cross-linking reagent, adipic acid dihydrazide (ADH), in the presence of reductant sodium cyanoborohydride in order to introduce reactive amino groups in intermediate B as shown in reaction (ii) below:

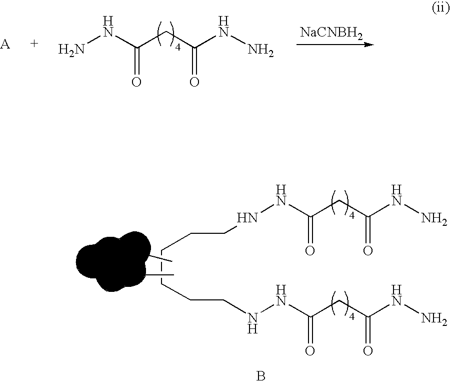

Many other reagents that are well known to those who are skilled in organic chemistry can transform aldehydes into amino moieties. These reagents can be used instead of or in addition to ADH. In this instance, the inventors discovered that the ADH moiety conveniently possesses buffering properties, which simplified the chemistry because no further linker or modification was necessary to introduce a buffer, as explained more fully below. Alternatives to ADH include, for instance, isophthalic dihydrazide (IDH) and sebacic dihydrazide (SDH). Thus, other synthetic strategies that present amino moieties, as in reaction (ii) above, should account for the need for a buffer, as detailed in a further embodiment below.

Finally, it should be noted that intermediate B is depicted as being doubly substituted with ADH moieities only for illustrative purposes. In practice, at least one and any number of additional aldehyde groups can be present for reaction with a reagent such as ADH to introduce at least one ADH or other similar moiety having an amino group.

Intermediate B was then treated with the well-known reagent N-succinimidy1-6-(3′-(2-pyridyldithio)-propionamido)-hexanoate, LC-SPDP, in order to introduce a convenient source of a disulfide unit in intermediate C that is capable of reacting with a thiol-substituted protein. The transformation is illustrated by reaction (iii) below:

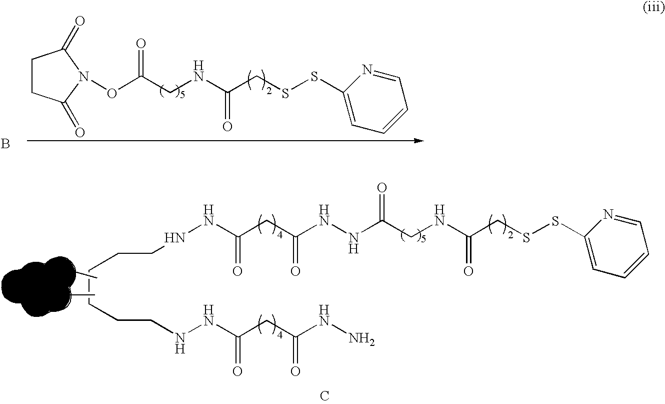

The reaction between B and LC-SPDP or another suitable source of a disulfide moiety may result in fewer than all amino moieties participating in the reaction, as shown above. Less than complete reaction is acceptable. What is important is that at least one amino moiety reacts so as to install a disulfide moiety, thereby allowing attachment to an appropriately modified protein.

Heterobifunctional cross-linking reagents other than LC-SPDP are well known and are suitable for use in reaction (iii) above, and they also provide a disulfide unit. These include sulfo N-succinimidy1-6-(3′-(2-pyridyldithio)-propionamido)-hexanoate (sulfo-LC-SPDP) and N-succinimidyl-3-(2-pyridyldithio)-propionate (SPDP). It is not necessary to use these particular reagents, so long as a protein ultimately can be attached to the particle. However, the reagents described above are convenient sources of disulfide moieties that are well-adapted for use in protein chemistry.

Finally, intermediate C was treated with a protein that has been reduced to display at least one sulfhydryl group, —SH, for reaction with the disulfide moiety in C, as shown in reaction (iv) below, to yield the final product D:

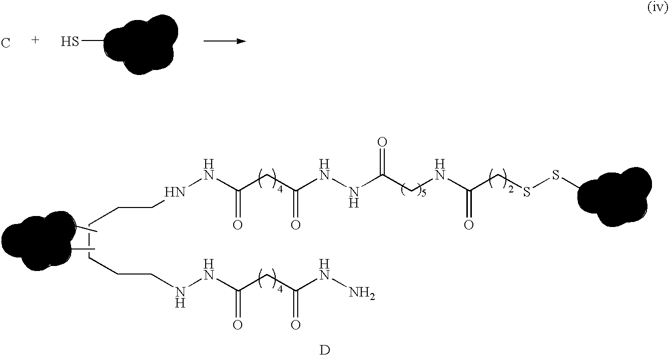

As mentioned above, it is not necessary to link the particle and protein with reagents that serve simultaneously as linkers and buffers, as illustrated by reactions (i)-(iv) above. It is sufficient that a protein ultimately is linked to the particle and that the product is buffered to pH of about 6- to about pH 8, as mentioned above.

Thus, in another embodiment, the particle is derivatized with moieties that separately confer buffering and linking capacities, respectively. For instance, reaction (v) below illustrates how intermediate A was reacted with an oligo-histidine and 1,4-diaminobutane to yield the mixed addition product E:

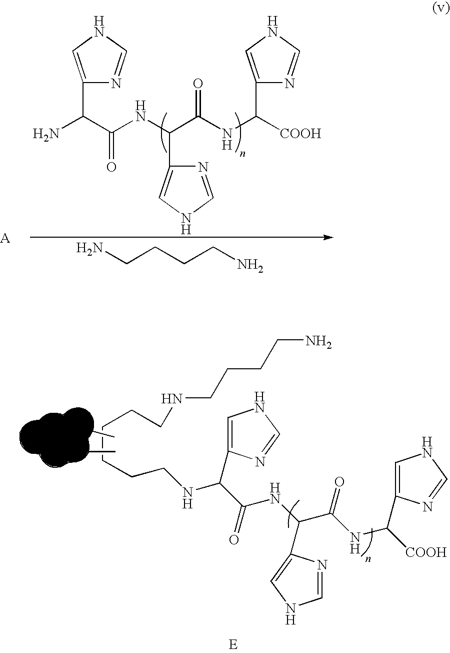

The oligo-histidine can be various lengths. What is important is that it confers buffering properties to the final product. Because the oligo-histidine terminates in a carboxyl group, it is not well-adapted for use in the synthetic methodologies described above for further reaction with reagents containing disulfide moieties. For this reason, the bifunctional diamine serves as a linker. In principle, any diamine is suitable for this purpose because it contains a requisite amino group to react with intermediate A as well as an amino group for further reaction with the disulfide-containing reagents. Typical diamines are primary amines because they are the most reactive.

In accordance with the general guidelines above, intermediate E was then treated with LC-SPDP to give intermediate F that contains a disulfide moiety, as illustrated in reaction (vi):

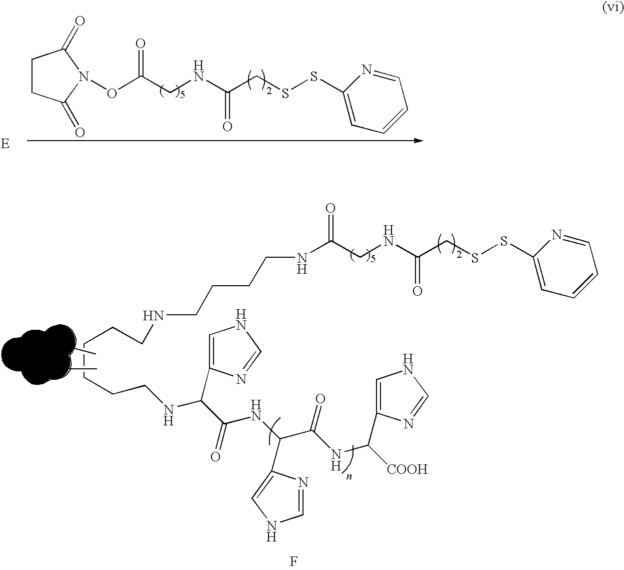

In some embodiments, as described above, other disulfide-containing reagents are used instead of LC-SPDP. Regardless of which reagent is selected, it follows from these methodologies that the presence of the oligo-histidine buffer effectively reduces the number of attachment points to the particle that will be available to an appropriately modified protein.

Thus, reaction of intermediate F with a sulfhydryl-modified polypeptide component yielded final product G, as depicted in reaction (vii) below:

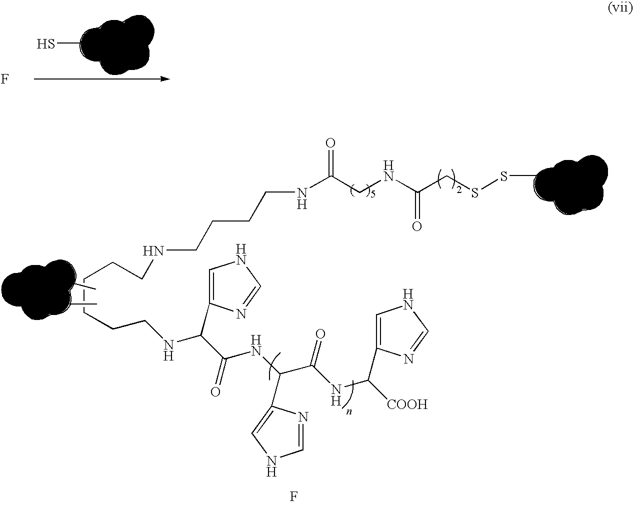

Ultimately, the polypeptide component is released intracellularly from the phagocytosed particle into the cytoplasm.

### Formulation

The compositions of the present invention may be formulated for mucosal administration (e.g., intranasal and inhalational administration) or percutaneous administration. The compositions of the invention can also be formulated for parenteral administration (e.g., intramuscular, intravenous, or subcutaneous injection), and injected directly into the patient and target cells of monocytic origin, like macrophages and dendritic cells. Thus, the compositions of the present invention may be administered just like a conventional vaccine. This also substantially reduces cost because of the lower level of skill required.

Formulations for injection may be presented in unit dosage form, e.g., in ampules or in multi-dose containers, optionally with an added preservative. The compositions may take such forms as suspensions, solutions or emulsions in oily or aqueous vehicles, and may contain formulatory agents such as suspending, stabilizing and/or dispersing agents. The composition of the present invention may also be formulated using a pharmaceutically acceptable excipient. Such excipients are well known in the art, but typically will be a physiologically tolerable aqueous solution. Physiologically tolerable solutions are those which are essentially non-toxic. Preferred excipients will either be inert or enhancing, but a suppressive compound may also be used to achieve a tolerogenic response.

### Therapeutic Methods

The compositions of the present invention attract phagocytic cells, such as cells of the mononuclear phagocyte system, including monocytes, macrophages, dendritic cells or immature dendritic cells. In the field of vaccination, cells of the mononuclear phagocyte system are considered “professional” antigen presenting cells and, thus, are the ideal target for vaccine delivery. It is well known that presentation of an antigen within an APC is vastly more effective in generating a strong cellular immune response than expression of this same antigen within any other cell type. Therefore, the ability of the compositions of the present invention to present a polypeptide component for display on an antigen presenting cell via class I MHC molecules dramatically enhances the efficacy of such a vaccine.

The compositions of the present invention can be used to develop CD8 T cell vaccines against viral, bacterial and parasitic infections, as well as cancer. The polypeptide component in the compositions of the present invention is delivered in an amount sufficient to provoke a CD8 T cell response.

The use of modified particles that can be phagocytosed, including yeast cell wall particles, present a number of advantages over conventional vaccine methodologies.

First, the compositions disclosed herein would obviate the need for attenuated live vaccines to obtain protective CD8 T cell immune responses without infectious agents because transfer of inactivated pathogen structures to the MHC-I pathway is mediated by the particle to be phagocytosed.

Second, the modified particle can accommodate a large amount of different cargo molecules, which together with the avid phagocytosis, ensures very effective transfer of antigens to the MHC-I pathway of professional antigen presenting cells. Furthermore, there is no restriction on the molecular size of antigenic structures, as the particle to be phagocytosed can accommodate whole inactivated virus particles. Additionally, the large surface area of the particle to be phagocytosed, such as zymosan, would allow for the attachment of compounds that potentiate the phagocyte response to the antigen, such as CpG motifs.

Third, zymosan for example, due to its inherent phagocyte stimulating capacity as a microbial compound, in itself should have potent adjuvant properties, which is a perquisite for the induction of a primary CD8 T cell response.

Fourth, because antigen uptake happens via the phagocytic route, it is to be expected that part of the antigenic material would be presented simultaneously via the MHC class II pathway, which would ensure induction of a concomitant CD4 T cell helper response, which in turn is required for a productive CD8 T cell response.

Lastly, ready to use vaccines may be prepared within a short period of time without the requirement for specialized equipment, provided appropriate antigenic material is available.

The compositions of the present invention can be used in both a prophylactic context as well as a therapeutic one. For a prophylactic vaccine, the polypeptide component as part of the compositions disclosed herein that is delivered to an antigen presenting cell is designed to trigger an immune response against the polypeptide/antigen. A therapeutic vaccine is also designed to provoke an immune response, but in individuals already affected with the disease or disorder. The present invention contemplates both prophylactic and therapeutic uses of the compositions disclosed herein.

The compositions of the present invention come into contact with phagocytic cells either in vivo or in vitro. Hence, both in vivo and ex vivo methods are contemplated.

As for in vivo methods, the compositions of the present invention are generally administered parenterally, usually intravenously, intramuscularly, subcutaneously or intradermally. They may be administered, e.g., by bolus injection or continuous infusion. In ex vivo methods, monocytic cells are contacted outside the body and the contacted cells are then parenterally administered to the patient.

### Examples

The following non-limiting examples are given by way of illustration only and are not to be considered limitations of this invention. There are many apparent variations within the scope of this invention.

### Example 1

### Antigenic Stimulation of B3Z Cells

The phagocytosis carrier system is based on yeast cell wall particles that have been chemically modified to allow escape from the phagosome and release of covalently attached cargo molecules into the cytoplasm. The technical feasibility of this approach is exemplified in an in vitro model system for the presentation of a model peptide structure derived from ovalbumin (OVA) to a reporter MHC-I restricted CD8 T cell hybridoma line (B3Z), recognizing the internal OVA peptide sequence SIINFEKL. See, Shastri, N., and Gonzalez, F. 1993. J. Immunol. 150:2724.

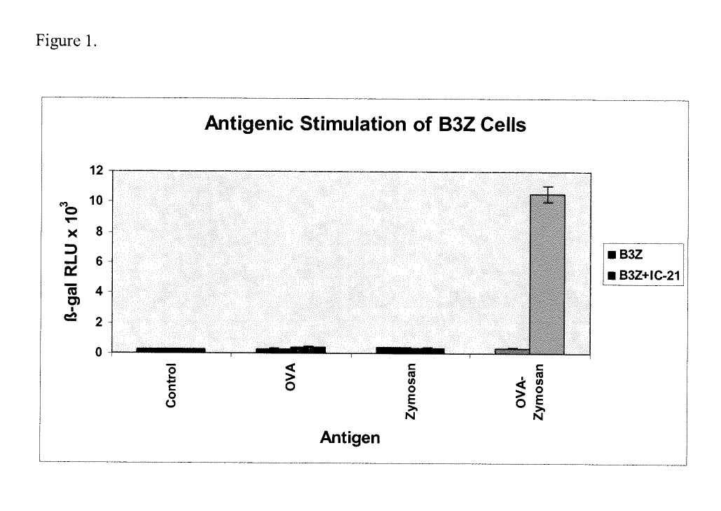

Results indicate that neither the soluble OVA nor the modified zymosan carrier alone caused stimulation of the B3Z hybridoma in the presence of MHC-I matched IC-21 presenting cells. Efficient antigen presentation, however, was observed with the zymosan-coupled OVA peptide (see induction of β-galactosidease reporter gene for T cell activation). Stimulation of B3Z cells was dependent on both phagocytosis and correct proteolytic processing of the zymosan-attached OVA because neither soluble nor zymosan coupled OVA induced β-gal reporter activity in the absence of IC-21 presenting cells. See FIG. 1.

These data demonstrate that efficient transfer of zymosan-coupled OVA to the cytoplasm as well as correct proteolytic processing via the proteosome and loading onto MHC-I molecules.

### Example 2

### Experimental Model Systems for Testing Vaccination Efficacy

Three principal experimental mouse model systems can be used to analyze the efficacy of zymosan-particle based vaccines.

In a first model system, a simple defined model protein antigen like OVA or β-gal is coupled to modified zymosan, induction of a cytolytic CD 8 T cell response is the readout, and established OVA or β-gal transfected, syngeneic tumor cell lines are the target cells for an in vitro assay of cytolytic T cell activity and an in vivo protection against tumor challenge. This model system allows the investigator to define the most basic parameters of vaccination, including dosage and vaccination schedule.

The second system model system is for vaccinating with complete viruses coupled to a modified yeast cell wall particle. As a model antigen, Moloney viruses would be attractive candidates because these retroviruses are known to rapidly cause sarcomas in various mouse strains which, after initial regression, prove lethal in most cases. As a readout system for this model, various established murine Moloney virus-transformed leukemia cell lines are used as model target cells both for in vitro cytolytic assays as well as in vivo tumor protection or tumor therapy assays. Furthermore, direct protection against virus-induced sarcoma formation may be used to assay vaccine efficacy in this model. This model system is also particularly attractive because retroviruses have been found to be involved in oncogenesis both in experimental animal systems and in humans, and in the case of HIV-1 are the causative agent of AIDS.

The third model system is for tumor vaccination with lysed tumor cells as the model antigen coupled to a modified yeast cell wall particle. The experimental readout system is in vitro cytolytic T cell activity against the tumor cells and both tumor protective and therapeutic efficacy in vivo.

What is claimed is:

1. An antigenic composition comprising (i) a polypeptide component, (ii) a buffering component, and (iii) a particle that can be phagocytosed, wherein the polypeptide component or a fragment thereof is ultimately presented on a class I MHC molecule.

2. The composition of claim 1, wherein the particle that can be phagocytosed is a biodegradable particle.

3. The composition of claim 2, wherein the particle that can be phagocytosed is a zymosan particle.

4. The composition of claim 1, wherein the buffering component is RCONHNH₂, oligohistidine, or polyethyleneimine.

5. A vaccine composition comprising (i) a polypeptide component, (ii) a buffering component, and (iii) a particle that can be phagocytosed, wherein the polypeptide component or a fragment thereof is delivered in an amount sufficient to provoke a CD8 T cell response, and the polypeptide component is ultimately presented on a class I MHC molecule.

6. The vaccine of claim 5, wherein the particle that can be phagocytosed is a biodegradable particle.

7. The vaccine of claim 6, wherein the particle that can be phagocytosed is a zymosan particle.

8. The vaccine of claim 5, wherein the buffering component has a buffering capacity in the range of about pH 6 to about pH 8.

9. The vaccine of claim 5, wherein the buffering component is RCONHNH₂, oligohistidine, or polyethyleneimine.

10. A method for efficient delivery of a polypeptide component to an antigen presenting cell comprising administering a composition comprising (i) a polypeptide component, (ii) a buffering component, and (iii) a particle that can be phagocytosed, wherein the polypeptide component, following administration, enters the cytosol from an endocytotic vesicle, and the polypeptide or a fragment thereof is presented on a MHC class I molecule.

11. The method of claim 10, wherein the particle that can be phagocytosed is a biodegradable particle.

12. The method of claim 11, wherein the particle that can be phagocytosed is a zymosan particle.

13. The method of claim 10, wherein the buffering component is RCONHNH₂, oligohistidine, or polyethyleneimine.

14. A composition for exogenous antigen presentation on class I MHC molecules comprising (i) a polypeptide component, (ii) a buffering component, and (iii) a particle that can be phagocytosed.

15. The composition of claim 14, wherein the particle that can be phagocytosed is a biodegradable particle.

16. The composition of claim 15, wherein the particle that can be phagocytosed is a zymosan particle.

17. The composition of claim 14, wherein the buffering component is RCONHNH₂, oligohistidine, or polyethyleneimine.
